(19) United States
(12) Patent Application Publication
(10) Pub. No.: US 2003/0004581 A1
(43) Pub. Date: Jan. 2, 2003

(54) Implantable prosthetic mesh system

(76) Inventor: Robert A. Rousseau, Ottsville, PA (US)

Correspondence Address: SELITTO, BEHR & KIM, 203 MAIN STREET, METUCHEN, NJ, 08840-2727, US

(21) Appl. No.: 09/892,340

(22) Filed: Jun. 27, 2001

(51) Int. Cl. A61F 2/02

(52) U.S. Cl. 623/23.74

(57) ABSTRACT

The present invention relates to a prosthetic mesh system adapted for implantation in a body. More particularly, the mesh system includes a biocompatible mesh layer. The mesh layer is flexible such that the mesh layer has a generally flat shape when it is in a first condition and a generally collapsed shape when it is in a second condition. The mesh layer has at least one ridge formed integrally therewith and projecting therefrom in a direction substantially perpendicular to the mesh layer when the mesh layer is in the first condition. The ridge is sized and shaped so as to facilitate the movement of the mesh layer from its collapsed shape to its flat shape.

### FIELD OF THE INVENTION

The present invention relates to a prosthetic mesh system and, more particularly, to a prosthetic mesh system adapted for implantation in a body.

### BACKGROUND OF THE INVENTION

In the past, surgical mesh layers have been used as underlay patches for repairing hernia defects (e.g., openings or holes formed in a wall of an organ, through which interior organs tend to protrude). For instance, U.S. Pat. Nos. 5,254,133 and 5,725,577 disclose patch-type prostheses placed in hernia defects to close off associated openings.

A typical underlay patch includes a generally flat mesh cut into a certain geometric (e.g., circular, oval, etc.) shape. After being inserted through a hernia defect in a folded or collapsed condition, the underlay patch is expanded into its flat condition so as to cover an opening formed in the hernia defect.

Various mechanism have been proposed for facilitating the intraoperative expansion of surgical meshes to their flat shape or condition. For instance, U.S. Pat. No. 5,254,133 discloses a surgical implantation device having resilient or shape memory segments attached thereto, while U.S. Pat. No. 5,368,602 discloses a patch of flexible surgical mesh material equipped with an elongated semi-rigid member attached thereto. In addition, European Patent Publication No. 0 544 485 B1 discloses a tissue aperture repair device having spokes or a stiffener member (see also U.S. Pat. No. 5,258,000), while U.S. Pat. No. 5,141,515 discloses an implanting device for use with patches.

Although the devices discussed above can be used intraoperatively to expand surgical meshes to their flat shape, they suffer from various problems. For instance, additional members or mechanisms separate from meshes are used in these devices, potentially increasing their production costs. Moreover, because of such additional members or mechanisms, the production and/or use of the devices are made complicated. In addition, the presence of an expansion member with stiffness greater than the surgical mesh results in less adaptability to the anatomy surrounding the hernia defect.

### SUMMARY OF THE INVENTION

The present invention overcomes the disadvantages and shortcomings of the prior art discussed above by providing a new and improved prosthetic mesh system adapted for implantation in a body. More particularly, the mesh system includes a biocompatible mesh layer. The mesh layer is flexible such that the mesh layer has a generally flat shape when it is in a first (i.e., expanded) condition and a generally collapsed shape when it is in a second (i.e., folded or compressed) condition. The mesh layer has at least one ridge formed integrally therewith and projecting therefrom in a direction substantially perpendicular to the mesh layer when the mesh layer is in the first condition. The ridge is sized and shaped so as to facilitate the movement of the mesh layer from its collapsed shape to its flat shape. In accordance with one feature of the present invention, the mesh system is adapted for use as a patch for repairing a hernia defect.

### BRIEF DESCRIPTION OF THE DRAWINGS

For a more complete understanding of the present invention, reference is made to the following detailed description of exemplary embodiments considered in conjunction with the accompanying drawings, in which:

FIG. 1 is a plan view of a surgical mesh system constructed in accordance with a first embodiment of the present invention;

FIG. 2 is a cross-sectional view, taken along section lines 2-2 and looking in the direction of the arrows, of the surgical mesh system shown in FIG. 1;

FIG. 3 is an enlarged view of a portion of the surgical mesh system shown in FIG. 2;

FIG. 4 is a schematic view of the surgical mesh system shown in FIGS. 1-3, the surgical mesh system being placed in a hernia defect;

FIG. 5 is a plan view of a surgical mesh system constructed in accordance with a second embodiment of the present invention;

FIG. 6 is a cross-sectional view, taken along section lines 6-6 and looking in the direction of the arrows, of the surgical mesh system shown in FIG. 5;

FIG. 7 is an enlarged view of a portion of the surgical mesh system shown in FIG. 6;

FIG. 8 is a plan view of a surgical mesh layer constructed in accordance with a third embodiment of the present invention; and

FIG. 9 is a cross-sectional view, taken along section lines 9-9 and looking in the direction of the arrows, of the surgical mesh layer shown in FIG. 8.

### DETAILED DESCRIPTION OF THE EXEMPLARY EMBODIMENTS

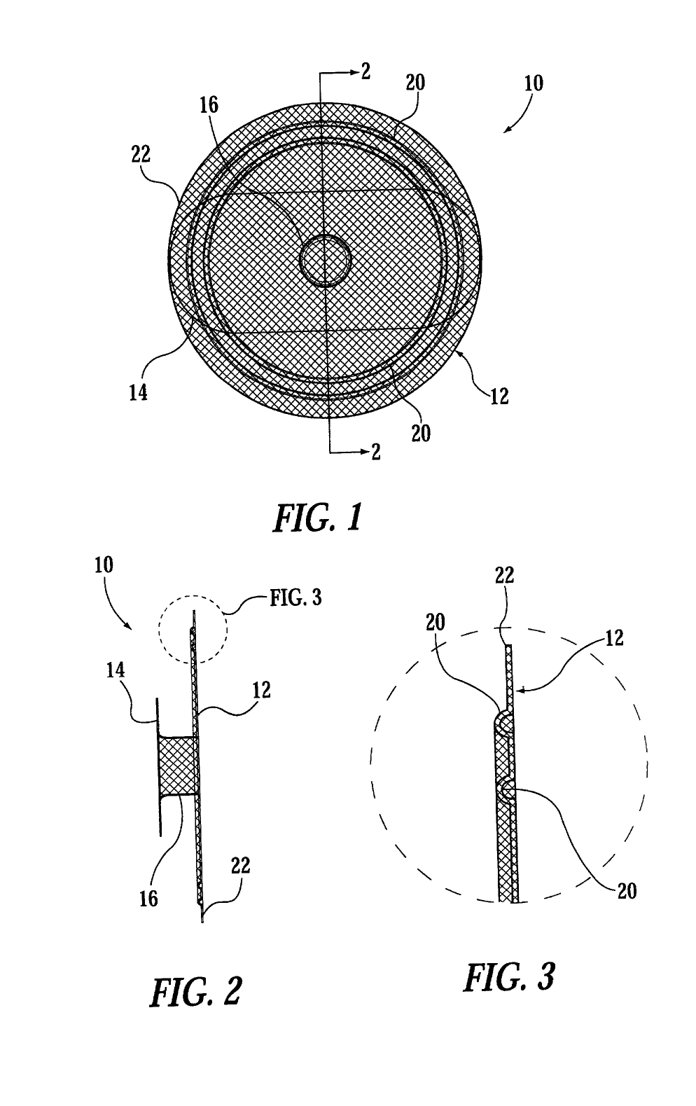

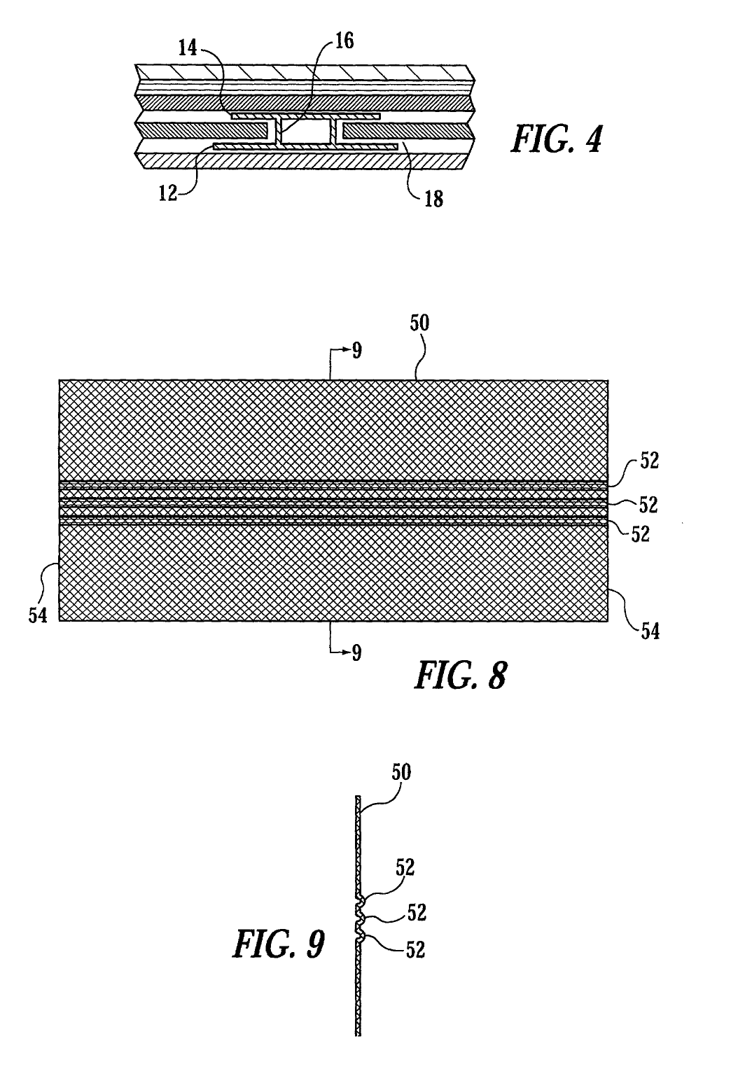

FIG. 1 shows a surgical mesh system 10 constructed in accordance with a first embodiment of the present invention. More particularly, the surgical mesh system 10 includes a pair of patches (i.e., layers) 12, 14 and a connecting member 16 connecting the patches 12, 14 to one another. The patches 12, 14 are sized and shaped so as to be positioned on opposing sides of an opening of a hernia defect 18 (see FIG. 4) in a conventional manner. The patches 12, 14 and the connecting member 16 are made preferably from a polypropylene mesh, such as the meshes marketed by Johnson & Johnson under the trademark “PROLENE”. Alternatively, other biocompatible materials, such as polytetraflouroethylene, can be used for the surgical mesh system 10. Accordingly, the patches 12, 14 are flexible such that they have a generally flat (i.e., planar) shape when they are in an expanded condition and a generally collapsed shape when they are in a compressed or folded condition. In this manner, the surgical mesh system 10 can be delivered to the hernia defect 18 in its collapsed shape.

With reference to FIGS. 1-3, the patch 12 is shaped so as to form therein a pair of concentric rings 20 located adjacent to the circumference 22 of the patch 12. More particularly, each of the rings 20 is in the form of a ridge and is part of the patch 12 (i.e., is integral with the patch 12). In other words, each of the rings 20 projects from the patch 12 in a direction substantially perpendicular to the patch 12 and hence is non-coplanar relative to same (i.e., the rings 20 have a raised three-dimensional geometrical shape, while the rest of the patch 12 is substantially flat) Although the rings 20 are preferably formed by a conventional thermo-forming process used in shaping surgical meshes, other processes can be used for shaping the patch so as to provide the rings 20 therein.

It should be appreciated that the rings 20 facilitate the expansion or movement of the patch 12 from its collapsed condition to its extended or flat condition intraoperatively. Without limiting the scope of the present invention, it is believed that when the patch 12 is bent out of its flat or planar shape, the three-dimensional geometrical shape of the rings 20 undergoes deformation, creating reactive force upon the entire patch 12. The reactive force causes the patch 12 to resume or return to its flat shape during intraoperative placement. As a result, the patch 12 is adapted to expand from its collapsed shape to its flat shape without the use of a separate or external member or without changing (i.e., increasing) the stiffness or rigidity of the basic mesh material of the patch 12. In this manner, the patch 12 maintains flexibility such that it can conform to anatomical structures adjacent to the hernia defect 18 without having certain portions with rigidity greater than the rest of the patch 12.

It should be noted that the surgical mesh system 10 can have numerous modifications and variations. For instance, the patch 14 can be provided with rings similar to the rings 20. Moreover, the rings 20 can be modified to have different geometric shape or orientation. By way of example, the patch 12 can be provided with ridges extending parallel to one another in a linear direction.

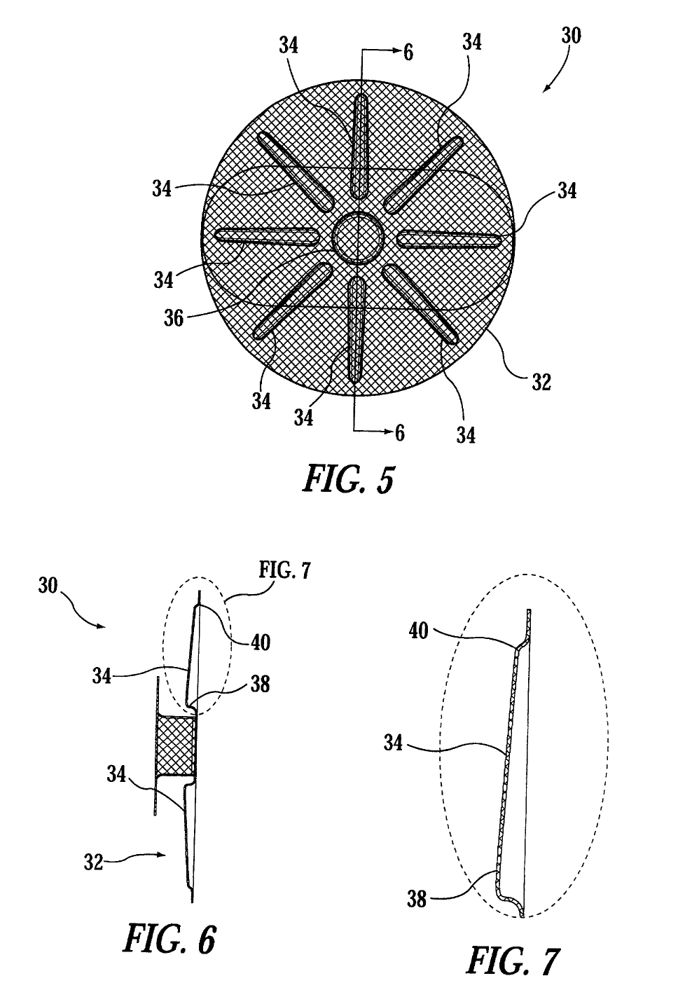

FIGS. 5-7 illustrate a surgical mesh system 30 constructed in accordance with a second embodiment of the present invention. More particularly, the surgical mesh system 30 has a construction identical to that of the surgical mesh system 10 shown in FIGS. 1-4, except as follows. The surgical mesh system 30 has a patch 32 provided with a plurality of ridges 34 extending radially outwardly from a center 36 of the patch 32. Each of the ridges 34 has an end 38, which is located adjacent to the center 36, and an end 40, which is located remote from the center 36. The ends 38 of the ridges 34 are provided with a height greater than the height at the ends 40 of the ridges 34 (see FIG. 7). Moreover, the ridges 34 taper as they extend from the ends 38 to the ends 40. Like the rings 20 of the surgical mesh system 10 shown in FIGS. 1-4, the ridges 34 facilitate the patch 32 to expand or move from its folded or collapsed condition to its flat condition.

It should be noted that the surgical mesh system 30 can have numerous modifications and variations. For instance, one or both of the rings 20 shown in FIGS. 1-3 can be formed in the patch 32 together with the ridges 34.

FIGS. 8 and 9 illustrate a surgical mesh layer 50 constructed in accordance with a third embodiment of the present invention. More particularly, the surgical mesh layer 50 is constructed as a single sheet and has a plurality of projecting ridges 52 extending linearly between opposing ends 54 of the surgical mesh layer 50. Alternatively, other geometrical patterns or shapes (e.g., crisscrossing patterns) can be used for the ridges 52. The ridges 52 perform the same basic function as the rings 20 and ridges 34 of the surgical mesh systems 10, 30 shown in FIGS. 1-4 and FIGS. 5-7, respectively. While the surgical mesh layer 50 is particularly suitable for use as a patch for repairing hernia defects, it can be used for other medical applications (e.g., pelvic floor reconstruction, mesh tamponading of organs, etc.).

Two samples of the surgical mesh layer 50 (i.e., Samples A and B having ridges extending in wale and course directions, respectively) were tested using a cantilever test method for the purpose of measuring their flexural rigidity. A sample of a surgical mesh layer without ridges (i.e., Sample C) was also tested as control. According to this experiment, the flexural rigidity of Sample A was about 6781 mg*cm, while the flexural rigidity of Sample B was about 7335 mg*cm. The flexural rigidity of Sample C (control) was measured to be about 624 mg*cm.

It will be understood that the embodiments described herein are merely exemplary and that a person skilled in the art may make many variations and modifications, including those discussed above, without departing from the spirit and scope of the invention. All such variations and modifications are intended to be included within the scope of the invention as defined in the appended claims.

I claim:

1. A prosthetic mesh system adapted for implantation in a body, comprising a biocompatible mesh layer, said mesh layer being flexible such that said mesh layer has a generally flat shape when it is in a first condition and a generally collapsed shape when it is in a second condition, said mesh layer having at least one ridge formed integrally therewith and projecting therefrom in a direction substantially perpendicular to said mesh layer when said mesh layer is in said first condition, said at least one ridge being sized and shaped so as to facilitate the movement of said mesh layer from its said collapsed shape to its said flat shape.

2. The prosthetic mesh system of claim 1, wherein said at least one ridge is formed by a thermo-forming process.

3. The prosthetic mesh system of claim 2, wherein said at least one ridge is sized and shaped such that said mesh layer is expandable from its said collapsed shaped to its said flat shape after being implanted in a body.

4. The prosthetic mesh system of claim 3, wherein said mesh layer is sized and shaped so as to be used as a patch for repairing a hernia defect.

5. The prosthetic mesh system of claim 4, wherein said mesh layer has a circular shape.

6. The prosthetic mesh system of claim 5, wherein said at least one ridge includes a plurality of ridges formed in said mesh layer.

7. The prosthetic mesh system of claim 6, further comprising another mesh layer and a connecting member connecting said another mesh layer to said mesh layer.

8. The prosthetic mesh system of claim 7, wherein each of said ridges has a ring shape.

9. The prosthetic mesh system of claim 8, wherein said ridges are arranged in a concentric manner.

10. The prosthetic mesh system of claim 7, wherein said ridges extend radially outwardly from a center of said mesh layer.

11. The prosthetic mesh system of claim 6, wherein each of said ridges has a ring shape.

12. The prosthetic mesh system of claim 11, wherein said ridges are arranged in a concentric manner.

13. The prosthetic mesh system of claim 6, wherein said ridges extend radially outwardly from a center of said mesh layer.

14. The prosthetic mesh system of claim 3, wherein said at least one ridge includes a plurality of ridges formed in said mesh layer.

15. The prosthetic mesh system of claim 14, wherein said mesh layer has opposing ends, said ridges extending linearly between said opposing ends.
